(19) United States
(12) Patent Application Publication
(10) Pub. No.: US 2021/0038753 A1
(43) Pub. Date: Feb. 11, 2021

(54) BIOLOGICAL STERILIZATION INDICATOR WITH STERILANT RESISTANCE MODULATOR

(71) Applicant: 3M INNOVATIVE PROPERTIES COMPANY, SAINT PAUL, MN (US)

(72) Inventors: SWARNALATHA SWAMINATHAN, WOODBURY, MN (US); ASSUMPTA A. G. BENNAARS-EIDEN, WOODBURY, MN (US); FRANCOIS AHIMOU, WOODBURY, MN (US); WILLIAM E. FOLTZ, COTTAGE GROVE, MN (US)

(73) Assignee: 3M INNOVATIVE PROPERTIES COMPANY, SAINT PAUL, MN (US)

(21) Appl. No.: 17/077,455

(22) Filed: Oct. 22, 2020

Related U.S. Application Data
(63) Continuation of application No. 15/509,888, filed on Mar. 9, 2017.
(60) Provisional application No. 62/062,285, filed on Oct. 10, 2014.

(51) Int. Cl. A61L 2/28; C12M 1/12; C12Q 1/22; G01N 31/22; C12M 1/34

(52) U.S. Cl. CPC A61L 2/28; C12M 37/06; C12M 1/34; G01N 31/226; C12Q 1/22

(57) ABSTRACT

A self-contained biological sterilization indicator is provided. The self-contained biological sterilization indicator includes an outer container having at least one liquid-impermeable wall and an interior volume; a sealed, openable, liquid-impermeable inner container enclosing a predetermined volume of an aqueous medium; a dry coating that comprises i) a plurality of viable test microorganisms useful to detect exposure to an oxidative sterilant and ii) an effective amount of a sterilant-resistance modulator; and a pathway that permits vapor communication between the interior volume and an atmosphere outside the outer container. The inner container and the dry coating are disposed in the interior volume. The modulator comprises an amino acid. The effective amount causes an increase in sensitivity of the test microorganisms to the oxidative sterilant relative to an otherwise-identical dry coating that lacks the effective amount.

### CROSS REFERENCE TO RELATED APPLICATIONS

This application is a continuation of U.S. patent application Ser. No. 15/509,888, filed Mar. 9, 2017, which is a national stage filing under 35 U.S.C. 371 of PCT/US2015/054252, filed Oct. 6, 2015, which claims priority to U.S. Provisional Patent Application No. 62/062,285, filed Oct. 10, 2014, the disclosures of which are incorporated by reference in their entirety herein.

### BACKGROUND

Sterilization indicators (also referred to as biological sterilization indicators) provide a means for determining whether a sterilizing machine, such as those used to sterilize surgical instruments in hospitals, is functioning properly and killing microorganisms that are present in the sterilization chamber during a sterilization procedure.

Sterilization indicators, including self-contained sterilization indicators, are recognized in the art as providing an accurate and precise means for testing the effectiveness of a sterilization procedure. Conventional sterilization indicators gauge the effectiveness of a sterilization procedure by monitoring the survival of a test microorganism contained within the sterilization indicator that is many times more resistant to the sterilization process than most organisms that would ordinarily be present by natural contamination. The sterilization indicator is exposed to a sterilization cycle and then incubated under conditions that will promote the growth of any surviving test microorganisms. If the sterilization cycle fails, the sterilization indicator generates a detectable signal indicating that the biological specimen survived. The detectable signal is commonly an indication such as a color change or the emission of a luminescent or fluorescent signal.

One well-known type of self-contained sterilization indicator employs spores from bacteria or fungi, which are very resistant to sterilization, to test the effectiveness of a sterilization procedure. A typical self-contained sterilization indicator has an outer container and a sealed inner container. A bacteria impermeable, gas transmissive covering on the outer container allows sterilant to enter the outer container during a sterilization procedure. Live spores on a carrier are located between the walls of the outer container and the inner container. The inner container contains a growth medium that stimulates the growth of live spores. During the sterilization procedure, sterilant enters the outer container through the cap and contacts the spores within the carrier. After the sterilization procedure, the inner container is crushed, releasing the growth medium and bringing it into contact with the spores. The indicator is then incubated under conditions that stimulate spore growth. If the sterilization procedure is ineffective, surviving spores will grow out and cause a pH indicator in the growth medium to change color, indicating that the sterilization cycle failed to kill the test population of microorganisms and may have failed to kill contaminating microorganisms present in the sterilizer load. Although sterilization indicators that rely on the growth of spores are accurate, they are slow, commonly requiring between 1 and 7 days to provide final results.

In contrast to sterilization indicators that measure spore growth alone, enzyme indicators provide a rapid answer, often in a matter of a few hours. Such indicators measure the effectiveness of a sterilization procedure by measuring the activity of an enzyme whose activity is correlated with the destruction of contaminating microorganisms during a sterilization procedure. If the sterilization procedure works properly, the enzyme is inactivated during the procedure and there is no detectable change following incubation. However, if the sterilization procedure is ineffective, the enzyme is not inactivated and will react with the substrate to form a detectable product. The enzyme-substrate product may be detectable as a color change or as a fluorescent or luminescent signal.

Dual rapid-readout indicators are self-contained sterilization indicators that test the effectiveness of a sterilization procedure by measuring both enzyme activity and spore growth following exposure to a sterilization procedure. The enzyme system gives a rapid indication of the effectiveness of a sterilization cycle, which is then confirmed by measurement of spore outgrowth over a longer period of time. In a dual rapid-readout indicator, the live spores utilized in the spore outgrowth portion of the indicator may also serve as the source of active enzyme for the enzyme activity portion of the assay. The rapid enzyme test measures the activity of an enzyme that is associated with the spores, and the spores themselves are then incubated to encourage the outgrowth of any spores that survived the sterilization procedure. 3M ATTEST™ 1291 and 1292 Rapid-readout Biological indicators, available from 3M Company, St. Paul, Minn., are dual rapid-readout indicators that test the effectiveness of a sterilization cycle by measuring both the activity of an enzyme associated with Geobacillus stearothermophilus (formerly known as Bacillus stearothermophilus)spores in the indicator and the survival of the spores themselves.

### SUMMARY

The present disclosure provides articles and methods for determining the efficacy of a sterilization process. The articles comprise a dry coating that comprises i) a plurality of viable test microorganisms useful to detect exposure to an oxidative sterilant and ii) an effective amount of a sterilant-resistance modulator. The effective amount of sterilant resistance modulator causes an increase in sensitivity of the biological indicators to the oxidative sterilant relative to an otherwise-identical dry coating that lacks the effective amount. Advantageously, the sterilant resistance modulators of the present disclosure are capable of lowering the resistance of the biological indicators to oxidative sterilants without having a substantial negative effect on rapid detection of an enzyme activity associated with the test microorganisms (e.g., the sterilant resistance modulator does not cause a substantial delay in the ability to detect the enzyme activity.

Advantageously, the modulators afford the ability to adjust the resistance of biological indicators to oxidative sterilants.

In one aspect the present disclosure provides a self-contained biological sterilization indicator. The self-contained biological sterilization indicator can comprise an outer container having liquid-impermeable walls and an interior volume; a sealed, openable, liquid-impermeable inner container enclosing a predetermined volume of an aqueous medium; a dry coating that comprises i) a plurality of viable test microorganisms useful to detect exposure to an oxidative sterilant and ii) an effective amount of a sterilant-resistance modulator; and a pathway that permits vapor communication between the interior volume and an atmosphere outside the outer container. The effective amount causes an increase in sensitivity of the biological indicators to the oxidative sterilant relative to an otherwise-identical dry coating that lacks the effective amount.

In another aspect, the present disclosure provides a biological sterilization indicator. The biological sterilization indicator can comprise a carrier and a dry coating disposed thereon.

The dry coating comprises i) a plurality of viable test microorganisms useful to detect exposure to an oxidative sterilant and ii) an effective amount of a sterilant-resistance modulator. The effective amount causes an increase in sensitivity of the biological indicators to the oxidative sterilant relative to an otherwise-identical dry coating that lacks the effective amount.

In any of the above embodiments, the sterilant resistance modulator can be selected from the group consisting of L-homocysteine, L-arginine, and L-histidine. In any of the above embodiments, the sterilant resistance modulator modulates resistance of the biological indicator to an oxidative sterilant or disinfectant comprising hydrogen peroxide, peracetic acid, ozone, chlorine dioxide, or combinations thereof.

In yet another aspect, the present disclosure provides a method of determining an efficacy of a sterilization process. The method can comprise providing the biological sterilization indicator of any one of the above embodiments; exposing the biological sterilization indicator to a sterilant in a sterilization process, wherein the sterilant is an oxidative sterilant; and detecting an indication whether at least one of the plurality of test microorganisms survived the sterilization process.

In yet another aspect, the present disclosure provides a method of determining an efficacy of a sterilization process. The method can comprise providing the self-contained biological sterilization indicator of any one of the above embodiments; exposing the self-contained biological sterilization indicator to a sterilant in a sterilization process, wherein the sterilant is an oxidative sterilant; and detecting an indication whether at least one of the plurality of test microorganisms survived the sterilization process.

In any of the above embodiments of the method, detecting an indication whether at least one microorganism of the plurality of test microorganisms survived the sterilization process can comprise detecting growth of the test microorganism. In any of the above embodiments of the method, detecting an indication whether at least one microorganism of the plurality of test microorganisms survived the sterilization process can comprise detecting a predetermined enzyme activity associated with the test microorganism.

Herein, the term “biological sterilization indicator” refers to a substrate (e.g., a carrier or a wall of a container) onto which a liquid volume comprising a predetermined quantity of test microorganisms is coated and subsequently dried (e.g., dehydrated) to a substantially water-free state. The phrase “substantially water-free” designates a coating which has a water content no greater than about the water content of a dehydrated coating once it has been permitted to equilibrate with the ambient environment.

Herein, the term “self-contained biological sterilization indicator” refers to a device comprising a test microorganism source (e.g., a biological sterilization indicator), a culture medium, and a means for forming a detectable indication of the failure of a sterilization procedure packaged together in a container that permits the test microorganism source, culture medium, and means for forming a detectable indication of the failure of a sterilization procedure to be combined without exposing the contents of the device to non-sterile surroundings.

Herein, a “porous” carrier means that sterilant can pass through the carrier under normal conditions of sterilization (such conditions are defined by the particular sterilization procedure).

Herein, “supported by” the carrier means that the test microorganisms may be disposed on the surface of the carrier (especially, if it is not porous) or distributed within a porous carrier.

Herein, “distributed within” a porous carrier means that the test microorganisms may be uniformly or nonuniformly distributed throughout at least a portion of the volume of a porous carrier (i.e., not only on its surface). “Distributed within” includes distributed throughout (and uniformly distributed throughout) the entire volume of the porous carrier.

Herein, a “test microorganism” refers to a microorganism commonly used to monitor the effectiveness of a sterilization procedure, such as Geobacillus stearothermophilus.

Herein, in the context of the material of which the carrier is made, the term “hydrophilic” means having a contact angle of zero (i.e., wetted by water). This hydrophobic material can be inorganic, organic, or a combination thereof.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

As used herein, “a,” “an,” “the,” “at least one,” and “one or more” are used interchangeably. Thus, for example, “a” test microorganism can be interpreted to mean “one or more” test microorganisms.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

Additional details of these and other embodiments are set forth in the accompanying drawings and the description below. Other features, objects and advantages will become apparent from the description and drawings, and from the claims.

### BRIEF DESCRIPTION OF DRAWINGS

FIG. 1 is an exploded view of one embodiment of a self-contained biological sterilization indicator according to the present disclosure.

FIG. 2 is a cross-sectional view of the device shown in FIG. 1.

FIG. 3 is an exploded view of an alternative embodiment of the self-contained sterilization indicator according to the present disclosure.

FIG. 4 is cross-sectional view of the device shown in FIG. 3.

FIG. 5 is a perspective view of one embodiment of a biological sterilization indicator according to the present disclosure.

FIG. 6 is an exploded view of the device shown in FIG. 5.

FIG. 7 is a perspective of an alternative embodiment of the self-contained biological sterilization indicator according to the present disclosure.

FIG. 8 is an exploded view of the device of FIG. 7.

### DETAILED DESCRIPTION

Before any embodiments of the present disclosure are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. It is to be understood that other embodiments may be utilized and structural or logical changes may be made without departing from the scope of the present disclosure.

The present disclosure generally relates to devices and methods for testing the effectiveness of a sterilization process. In particular, the present disclosure relates to devices that include a coating comprising a plurality of test microorganisms and a sterilant resistance modulator that functions to decrease the resistance of the biological indicators to an oxidative sterilant.

Biological sterilization indicators, including self-contained biological sterilization indicators, for testing the effectiveness of a sterilization procedure are provided, wherein the biological sterilization indicators include a plurality of substantially dry viable test microorganisms useful to detect exposure to an oxidative sterilant imbued with one or more sterilant resistance modulator, wherein the sterilant resistance modulator comprises an amino acid.

Biological sterilization indicators have been used previously to monitor the efficacy of sterilization systems. Biological sterilization indicators typically include a microorganism source with a predetermined concentration of live test microorganisms dried onto a carrier. The microorganism-impregnated carrier is placed in the loaded sterilization system and is subjected to a full sterilization process. Thereafter, the carrier is contacted with a sterile culture medium and incubated for a predetermined time at an appropriate temperature with a means for indicating the presence or absence of viable microorganisms (e.g., a pH indicator or an enzyme substrate that reacts with an enzyme to form a detectable product). At the end of the incubation period, the culture medium is examined to determine whether any test microorganisms survived the sterilization process. Microorganism survival means that the sterilization process was ineffective.

Self-contained biological sterilization indicators include the microorganism source, a culture medium, and the means for indicating the presence or absence of viable microorganisms packaged together in a way that permits the test microorganisms, the culture medium, and the means for indicating the presence or absence of viable microorganisms to be combined without exposing any of the aforementioned components to non-sterile surroundings. Examples of self-contained biological sterilization indicators are disclosed by Falkowski,et al. (U.S. Pat. No. 5,801,010) and Smith (U.S. Pat. No. 5,552,320). The microorganism source may produce a detectable (active) enzyme that is associated with the microorganisms when they are in a viable state. Conversely, the enzyme may be inactive when the microorganisms have been exposed to a sterilization process that is sufficient to render the microorganisms nonviable.

Generally, herein, a self-contained biological sterilization indicator for testing the effectiveness of a sterilization procedure includes: a container (e.g., a tube, sleeve, or ampoule) having at least one pathway (e.g., an opening) to allow a sterilant to enter the container during the sterilization procedure; an optional carrier contained within the container; test microorganisms (e.g., supported by the optional carrier), the test microorganism being a microorganism commonly used to monitor the effectiveness of a sterilization procedure; and means for forming a detectable indication of the failure of a sterilization procedure. Examples of self-contained biological sterilization indicators in which a test microorganism and corresponding sterilant resistance modulator of the present disclosure can be used include those described in International Publication Nos. WO 2012/061227 (Chandrapati et al.) or WO 2012/061226 (Smith et al.).

The biological sterilization indicators of the present disclosure can be used to measure spore growth only, enzyme activity only, or both enzyme activity and spore growth following exposure to a sterilization procedure. Preferred biological sterilization indicators measure the activity of an active enzyme whose activity is correlated with the survival of a test microorganism.

The test microorganisms supported by the carrier are selected such that they are inactivated (e.g., killed) by a sterilization procedure that is lethal to the test microorganism, but wherein the test microorganisms are not inactivated by a sterilization procedure that is sublethal to the test microorganism. Thus, the test microorganisms are inactivated as a result of an effective sterilization procedure. Conversely, a test microorganism that is not inactivated by the sterilization procedure provides a detectable indication as a result of an ineffective sterilization procedure. The detectable indication may involve an enzyme produced by the test microorganisms, the enzyme having an enzyme activity that is correlated with the survival of at least one test microorganism. The active enzyme is inactivated by a sterilization procedure that is lethal to the test microorganism. Conversely, the enzyme is not inactivated by a sterilization procedure that is sublethal to the test microorganism.

Test microorganisms useful in spore outgrowth indicators includes bacteria or fungi in either the spore or vegetative state. For biological sterilization indicators that include a rapid, enzyme-based readout, the test microorganisms include a source of active enzyme that is indigenous to the microorganism or is added to the microorganism by genetic engineering. In biological sterilization indicators of the present disclosure, the test microorganism is selected such that it is inactivated by a sterilization procedure that is lethal to the test microorganism, but wherein the test microorganisms may not be inactivated by a sterilization procedure that is sublethal to the test microorganism.

The carrier for the test microorganisms, if present, can be made of a material that is hydrophobic or hydrophilic. Such materials can be inorganic, organic, or combinations thereof. Carriers that include (or are prepared from) hydrophobic materials can be used with any indicator, whereas carriers that include (or are prepared from) hydrophilic materials are preferably used to monitor a sterilization procedure that uses hydrogen peroxide vapor phase. Examples of suitable hydrophobic materials include polypropylene, polyethylene, PET, polyurethane, nylon, polymer blends containing one or more of these polymers (e.g., with other hydrophobic polymers), or combinations thereof. Examples of suitable hydrophilic materials include glass. Other suitable materials for use as a carrier include glass fibers and metals (e.g., a stainless steel coupon) that do not substantially react with the sterilant.

Biological sterilization indicators (including self-contained biological sterilization indicators) of the disclosure may suitably be used to monitor the effectiveness of sterilization procedures that use hydrogen peroxide vapor phase (which may or may not include hydrogen peroxide plasma) For example, the biological sterilization indicators of the disclosure may be used to monitor the effectiveness of any of the hydrogen peroxide plasma sterilization procedures known in the art, including, for example, the procedures described in U.S. Pat. No. 4,643,876 (Jacobs et al.) and U.S. Pat. No. 4,756,882 (Jacobs et al.). Preferably, the biological sterilization indicator may be used to monitor the effectiveness of a hydrogen peroxide vapor phase sterilization procedure.

While aqueous hydrogen peroxide (H₂O₂) has a long history of use as a sterilant, the concept of vapor-phase hydrogen peroxide (VPHP) sterilization has been developed more recently. This process is a low temperature sterilization process that kills a wide range of microorganisms including bacterial endospore-forming bacteria commonly used as challenge organisms to evaluate and validate the effectiveness of sterilization cycles in hospitals. A major advantage of hydrogen peroxide is that a relatively short exposure (few minutes) to hydrogen peroxide is required in order to sterilize an object. Furthermore, at the end of a hydrogen peroxide sterilization process, only air and water remain in the chamber. Significantly, the novel features of the biological sterilization indicators described herein allow for the development of a rapid-readout hydrogen peroxide biological sterilization indicator.

In any embodiment of the biological sterilization indicator or the self-contained biological sterilization indicator according to the present disclosure, one or more sterilant resistance modulators is disposed in close proximity (e.g., on a carrier) with the test microorganisms.

Suitable examples of sterilant resistance modulators include amino acids such as L-homocysteine, L-arginine, L-histidine, and mixtures of any two or more of the foregoing amino acids.

One or more such sterilant resistance modulators can be disposed with the test microorganism or a component of the biological sterilization indicator such that both the test microorganism and the sterilant resistance modulator are exposed to the sterilant during the sterilization stage of the process.

In certain embodiments, the present disclosure provides a self-contained biological sterilization indicator for testing the effectiveness of a sterilization procedure, the indicator including: an outer container having liquid-impermeable walls and an interior volume;

contained within the outer container: a sealed, openable, liquid-impermeable inner container enclosing a predetermined volume of an aqueous medium, and a substantially-dry coating that comprises i) a plurality of viable test microorganisms useful to detect exposure to an oxidative sterilant and ii) an effective amount of a sterilant-resistance modulator, the effective amount causing an increase in sensitivity of the biological indicators to the oxidative sterilant relative to an otherwise-identical dry coating that lacks the effective amount; and a pathway that permits vapor communication between the interior volume and an atmosphere outside the outer container; wherein the modulator comprises an amino acid.

In any embodiment, a self-contained biological sterilization indicator according to the present disclosure comprises means for forming a detectable indication of the failure of a sterilization procedure. Exemplary means for forming the detectable indication are described herein. In any embodiment, the test microorganism can be distributed on and/or within (preferably, homogeneously) a carrier.

In sterilization indicators (including self-contained biological sterilization indicators) of the present disclosure, the carrier can include a material in a sheet form, whether it is porous or nonporous. In any embodiment, the test microorganisms may be distributed within a three-dimensional porous carrier. In this context, “distributed within” a three-dimensional porous carrier means that the test microorganisms may be uniformly or nonuniformly distributed throughout at least a portion of the volume of a three-dimensional porous carrier (as opposed to only on its surface). In any embodiment, the test microorganisms are distributed (more preferably, uniformly distributed) throughout the entire volume of a three-dimensional porous carrier. This can be accomplished by blending (e.g., in a laboratory blender) a sheet material, for example, to form a three-dimensional porous configuration and combining the test microorganisms before, during, or after blending.

Typically, the same quantity of test microorganisms is distributed within the three-dimensional porous carrier of the present disclosure, when compared to the quantity of test microorganisms disposed on conventional two-dimensional and/or nonporous carriers. This can result in a more even distribution of the test microorganisms (e.g., spores), thereby allowing the sterilant to penetrate into the three-dimensional porous carrier more thoroughly and have more uniform contact with the test microorganisms, compared to more densely packed and clustered test microorganisms on conventional carriers.

A porous carrier can be prepared and imbued with test microorganisms in a variety of ways, some of which are described in International Publication No. WO 2012/088064, which is incorporated herein by reference in its entirety. In one exemplary method, a nonwoven sheet material is converted to a three-dimensional structure by blending it in a laboratory blender to obtain a more voluminous structure (e.g., a three-dimensional structure, similar to that of a cotton ball). Alternatively, it can be chopped, melt-blown, or prepared using standard techniques for preparing nonwoven materials. This three-dimensional porous carrier is then removed from the blender and the desired test microorganisms (e.g., spores) are applied to the porous carrier.

Self-contained biological sterilization indicators of the present disclosure include a means for forming a detectable indication of the failure of a sterilization procedure. For example, self-contained biological sterilization indicators of the present disclosure can include a means for forming an enzyme-modified product (e.g., formed from reaction of enzyme substrate with active enzyme associated with the test microorganism) that provides a detectable indication of the failure of a sterilization procedure. This is typically referred to as the enzymatic activity test. This detectable indication of the failure of a sterilization procedure preferably includes a detectable fluorescence, luminescence, and/or chromogenic indication. These indications are preferably used for a quick enzymatic response in a rapid-readout biological sterilization indicator. In this context, “rapid-readout” means that a detectable signal is developed in less than 24 hours, and preferably within 8 hours or less.

In any embodiment, a self-contained biological sterilization indicator of the present disclosure may include means for forming a detectable indication wherein the detectable indication is associated with a by-product of microbial metabolism. In these embodiments (e.g., dual read-out biological sterilization indicators and biological sterilization indicators based solely on spore growth), the detectable indication of the failure of a sterilization procedure may include, for example, a detectable pH indication. The use of pH indication occurs upon growth of spores typically after 24 hours, and often after 7 days. In dual read-out biological sterilization indicators, this provides a mechanism for verifying the reliability of the rapid-readout. Generally, the pH indicator is one suitable for identification of acid formation, such as bromocresol purple, for example. This provides evidence of the stability and/or reliability of the readout obtained from the fluorescence, luminescence, and/or chromogenic indication, which are used for a quick enzymatic response. This is referred to as the spore outgrowth test.

In such embodiments in which spore outgrowth is evaluated (e.g., in a spore outgrowth indicator), after the sterilization procedure, the spores are brought into contact with growth medium (e.g., soybean casein digest optionally with a pH indicator). For example, a sealed, openable, liquid-impermeable inner container containing growth medium is crushed by compressing an outer container, releasing the growth medium and bringing it into contact with the test microorganism (optionally, supported by a carrier) in the outer container. The self-contained biological sterilization indicator is then incubated under conditions that stimulate growth of the test microorganism. If the sterilization procedure is ineffective, surviving test microorganisms will grow and their metabolic activity can cause a pH indicator in the growth medium to change color (e.g., as a result of acidic by-products formed from growing test microorganisms). This indicates that the sterilization cycle failed to kill the test population of microorganisms and may have failed to kill contaminating microorganisms present in the sterilizer load. Although self-contained biological sterilization indicators that rely on the growth of test microorganisms (e.g., spores) are accurate, they are slow, commonly requiring between 1 and 7 days to provide a final result.

In any embodiment of a method of making a self-contained biological sterilization indicator discussed above, the step of placing one or more components for forming a detectable indication of the failure of a sterilization procedure in the outer container can include placing an inner container including a growth medium that facilitates the growth of live test microorganisms (e.g., spores) and a pH indicator.

In certain embodiments of a method of making a self-contained biological sterilization indicator discussed above, the step of placing one or more components for forming a detectable indication of the failure of a sterilization procedure in the outer container includes placing an inner container including an enzyme substrate that reacts with an active enzyme associated with the test microorganism to form a detectable enzyme-substrate product.

In any embodiment, a self-contained biological sterilization indicator of the present disclosure includes: an outer container having at least one liquid-impermeable wall and an interior volume; a sealed, openable, liquid-impermeable inner container enclosing a predetermined volume of an aqueous medium; a substantially-dry coating, wherein the dry coating comprises i) a plurality of viable test microorganisms useful to detect exposure to an oxidative sterilant and ii) an effective amount of a sterilant-resistance modulator; and a pathway that permits vapor communication between the interior volume and an atmosphere outside the outer container; wherein the inner container and the dry coating are disposed in the interior volume; wherein the modulator comprises an amino acid; wherein the effective amount causes an increase in sensitivity of the biological indicators to the oxidative sterilant relative to an otherwise-identical dry coating that lacks the effective amount.

In any embodiment, the openable inner container (e.g., a tube, sleeve, or ampoule) is impermeable to a sterilant (e.g., vapor-phase hydrogen peroxide and/or plasma-phase hydrogen peroxide) under conditions that are used in sterilization procedures. In any embodiment wherein, after exposed to a sterilization process, the survival of the test microorganisms are evaluated for growth of surviving test microorganisms, the inner container includes a growth medium that facilitates growth of viable test microorganisms. The inner container is adapted (e.g., fabricated using a frangible material) so that it may be opened to allow contact between the growth medium and the test microorganisms.

In any embodiment wherein, after exposed to a sterilization process, the survival of the test microorganisms is determined by analyzing the test microorganisms for the presence of an active enzyme (e.g., an enzyme that is synthesized by viable test microorganisms). For example, in these embodiments, the inner container includes a substrate that reacts with the active enzyme. In these embodiments, the inner container of the self-contained biological sterilization indicator is adapted (e.g., fabricated using a frangible material) so that it may be broken to allow the enzyme substrate to react with the active enzyme to form an enzyme-modified product that provides a detectable indication of the failure of a sterilization procedure.

In any embodiment of a self-contained biological sterilization indicator of the present disclosure, preferably the outer container is compressible and the inner container is adapted so that it may be broken by compressing the outer container. Alternatively, the outer container may or may not be compressible and the inner container is adapted so that it may be broken by pushing down a cap to compress the inner container against an element (e.g., a sleeve) with prongs such that the inner container breaks upon being pushed into the prongs.

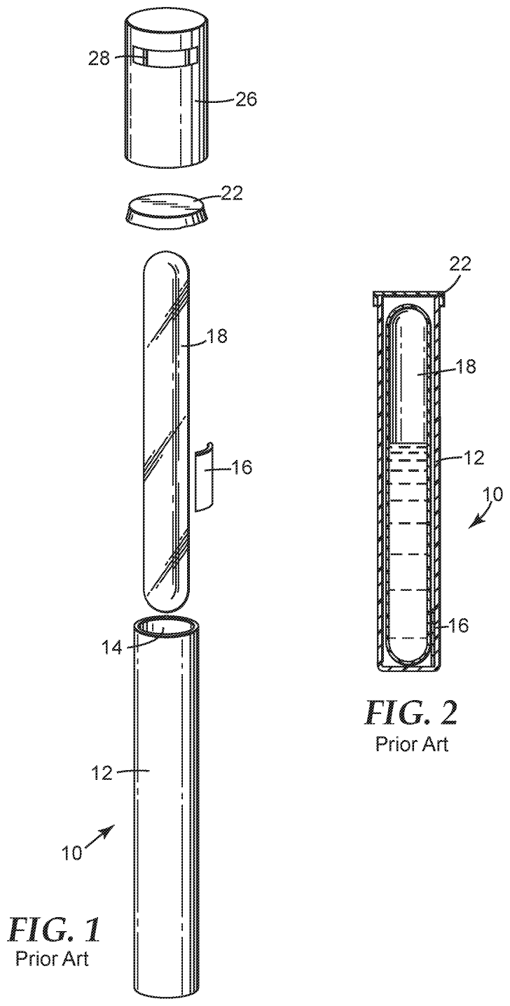

One embodiment of an exemplary self-contained biological sterilization indicator of the present disclosure is shown in FIGS. 1 and 2. The self-contained biological sterilization indicator 10 includes nesting containers that separate the various components of the system from each other until after the sterilization cycle is complete. The self-contained biological sterilization indicator 10 includes an outer container 12 (shown here in the form of a tube with an open end 14, although other types of containers can be used as would be understood by one of skill in the art), a sealed inner container 18 (shown here in the form of a sealed tube or ampoule, although other types of sealed containers can be used as would be understood by one of skill in the art) and a vented cap 26. Outer container 12 defines an interior volume and is preferably made of a plastic material (e.g., polyethylene, polypropylene). Inner container 18 is made of glass or some other liquid-impermeable frangible material. An optional closure member 22 is preferably a bacteria-impermeable, vapor-transmissive barrier that fits over the open end 14 of outer container 12.

An optional carrier 16 includes (i.e., supports) a substantially water-free dry coating (not shown) that comprises a plurality of test microorganisms and one or more sterilant resistance modulator according to the present disclosure. The carrier 16 is disposed in the inner volume of the outer container 12 (e.g., in a space between the inner container 18 and the outer container 12). In FIGS. 1 and 2 the carrier 16 is a strip of material (e.g., the flat carrier strip, made for example, of polymeric film. In any embodiment wherein the means for forming a detectable indication of the failure of a sterilization procedure comprises an enzyme substrate, the inner container 18 may contain an enzyme substrate that reacts with active enzyme associated with the test microorganism to create a detectable signal if the sterilization procedure is ineffective. In any embodiment wherein the means for forming a detectable indication of the failure of a sterilization procedure comprises an indicator for detecting microbial growth, inner container 18 also includes a growth medium for the test microorganisms that are supported by carrier 16 that survive, wherein spore outgrowth creates a detectable signal (e.g., turbidity, pH change) if the sterilization procedure is ineffective.

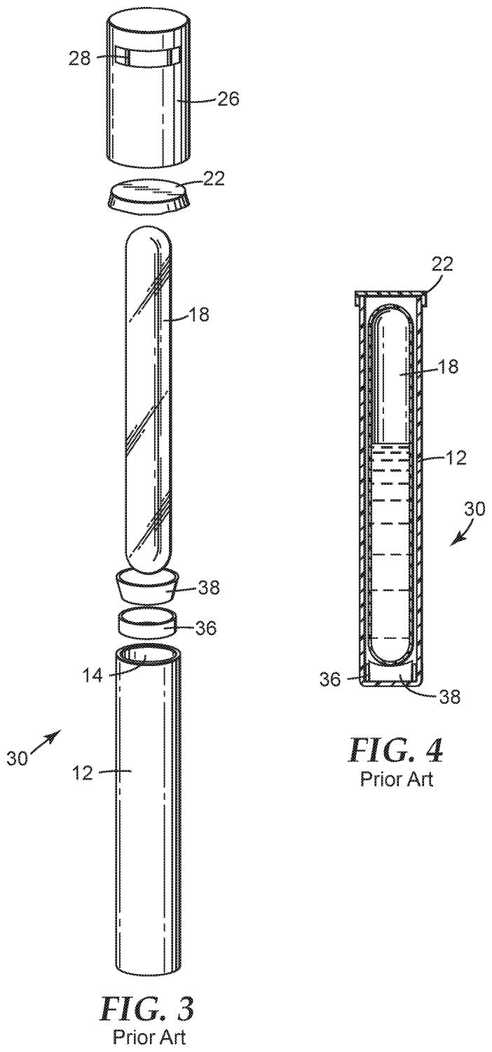

FIGS. 3 and 4 show an alternative embodiment, in which self-contained biological sterilization indicator 30 includes carrier 36 located in the interior volume of the outer container 12 proximate the closed end of the container, and a barrier 38 is situated between the carrier 36 and the inner container 18.

In FIGS. 3 and 4 the carrier 36 is a strip of material (e.g., a polymeric film). As described in the embodiments of FIGS. 1 and 2, in any embodiment wherein the means for forming a detectable indication of the failure of a sterilization procedure comprises an enzyme substrate, the inner container 18 may contain an enzyme substrate that reacts with active enzyme associated with the test microorganism to create a detectable signal if the sterilization procedure is ineffective. In addition, in any embodiment wherein the means for forming a detectable indication of the failure of a sterilization procedure comprises an indicator for detecting microbial growth, inner container 18 also includes a growth medium for the test microorganisms that are supported by carrier 16 that survive, wherein spore outgrowth creates a detectable signal (e.g., turbidity, pH change) if the sterilization procedure is ineffective.

Alternatively, in any embodiment, a carrier as described herein can be used advantageously without a barrier. For example, a carrier that includes a hydrophobic material (e.g., a hydrophobic polymeric film or nonwoven web) can function as both a carrier and a barrier.

Barrier 38 serves to isolate the carrier 36 from the inner container 18. Barrier 38 is preferably made from a hydrophobic material so that a product of a reaction between an active enzyme (produced by the test microorganisms) and a corresponding enzyme substrate, for example, concentrates proximate the porous carrier and does not diffuse rapidly throughout the entire inner volume of the outer container 12. Maintaining a higher concentration of the product of the active enzyme in the lower portion of the self-contained biological sterilization indicator enables the product, whether it is luminescent or colored, for example, to be detected after a shorter period of incubation than would be the case if the product was allowed to diffuse throughout the entire inner volume of the outer container 12. Preferred devices which incorporate a barrier 38 provide reliable information on sterilization efficacy within about 10 minutes.

The self-contained biological sterilization indicator configuration shown in FIGS. 3 and 4 with a barrier 38 is often used in a hydrogen peroxide vapor sterilization procedure. The barrier 38 is preferably a disc of polypropylene blown microfiber material having a weight of 200 g/sq. meter, commercially available as “THINSULATE 200-B brand Thermal Insulation” from 3M Company, St. Paul, Minn. Again, however, if the carrier is a hydrophobic material as described herein, the barrier is not needed, even in a hydrogen peroxide sterilization procedure. Also, when the self-contained biological sterilization indicator is to be used to monitor hydrogen peroxide procedures (whether using the embodiment shown in FIGS. 1 and 2 or FIGS. 3 and 4), closure member 22 is preferably made of a high-density fiber material, such as TYVEK high-density polyethylene fiber material, commercially available from E.I. du Pont de NeMours and Co., Wilmington, Del.

Referring back to the self-contained biological sterilization indicator of FIGS. 1 and 2, during a typical sterilization procedure sterilant enters the outer container 12 through the vents 28 on cap 26 (126) and contacts the test microorganisms (not shown) supported by carrier 16 but does not contact the contents in the sealed inner container 18 (e.g., enzyme substrate solution and/or growth medium). Thus, the vents 28 form a pathway that permits vapor communication between the interior volume and an atmosphere outside the outer container 12.

After the self-contained biological sterilization indicator of any embodiment of the present disclosure is exposed to a sterilization process, the sides of the outer container 12 can be compressed, breaking the inner container 18 and bringing the contents of the inner container 18 and the test microorganisms supported by carrier 16 into contact with each other. The self-contained biological sterilization indicator is then incubated for a period of time sufficient for any surviving test microorganisms to form a detectable indication. For example, if the test microorganisms produce an active enzyme, incubation occurs for a sufficient time for the active enzyme to react with the enzyme substrate to form a product that produces a detectable signal, such as luminescence, fluorescence or a color change, indicating that the sterilization procedure may have been ineffective.

In a preferred embodiment of the biological sterilization indicator 10 of the disclosure, the test microorganisms supported by carrier 16 are a source of an active enzyme. Preferably, the source of an active enzyme is a live test microorganism, such as a bacterial or fungal spore. In the most preferred embodiment, spores are the source of active enzyme, and the biological sterilization indicator 10 is a dual rapid-readout indicator that monitors the effectiveness of a sterilization procedure by measuring both enzyme activity and test microorganism outgrowth. In this embodiment, the inner container 18 contains a nutrient medium to facilitate growth of the test microorganisms and an enzyme substrate. After the self-contained biological sterilization indicator is exposed to a sterilization process, the inner container 18 is broken; thereby contacting the carrier 16 (and the test microorganisms thereon) with its contents; and the self-contained biological sterilization indicator is incubated for a period of time. The product of the enzyme reaction, if present after incubation, produces observable (e.g., visible) results within a few hours, and the growth of test microorganisms is typically observable within 7 days.

The theory underlying the operation of enzyme indicators is that the inactivation of the enzyme will be correlated with the death of test microorganisms in the biological sterilization indicator. The enzyme selected for use in a biological sterilization indicator must be at least as resistant to a sterilization procedure as microorganisms that are likely to be present as contaminants, and preferably more resistant than such microorganisms. The enzyme should remain sufficiently active to react with the corresponding enzyme substrate to form a detectable product after a sterilization cycle that fails to kill contaminating microorganisms, yet be inactivated by a sterilization cycle that kills contaminating microorganisms.

Enzymes that are suitable for use in the biological sterilization indicators of the disclosure are described in U.S. Pat. No. 5,252,484 (Matner et al.) and U.S. Pat. No. 5,073,488 (Matner et al.). Suitable enzymes include enzymes derived from spore-forming microorganisms, such as Geobacillus stearothermophilus and Bacillus atrophaeus (formerly known as Bacillus subtilis). Enzymes from spore-forming microorganisms that are useful in the biological sterilization indicators of the disclosure include beta-D-glucosidase, alpha-D-glucosidase, alkaline phosphatase, acid phosphatase, butyrate esterase, caprylate esterase lipase, myristate lipase, leucine aminopeptidase, valine aminopeptidase, chymotrypsin, phosphohydrolase, alpha-D-galactosidase, beta-D-galactosidase, tyrosine aminopeptidase, phenylalanine aminopeptidase, beta-D-glucuronidase, alpha-L-arabinofuranosidase, N-acetyl-B-glucosaminodase, beta-D-cellobiosidase, alanine aminopeptidase, proline aminopeptidase and a fatty acid esterase, derived from spore forming microorganisms.

When a test microorganism is used as the source of active enzyme, the method of the present disclosure may include the step of incubating any of the microorganisms which remain viable, following the completion of the sterilization cycle, with an aqueous nutrient medium. Inclusion of this step confirms by conventional techniques whether the sterilization conditions had been sufficient to kill all of the microorganisms in the indicator, indicating that the sterilization conditions had been sufficient to sterilize all of the items in the sterilizer. If growth of the microorganism is used in a conventional manner to confirm the results of the enzyme test, the microorganism should be one which is conventionally used to monitor sterilization conditions. These conventionally used microorganisms are generally many times more resistant to the sterilization process being employed than most organisms encountered in natural contamination.

Preferred microorganisms, which may be utilized as the test microorganism are bacteria or fungi in either the spore or vegetative state. The bacterial spore is recognized as the most resistant form of microbial life. It is the life form of choice in all tests for determining the sterilizing efficacy of devices, chemicals and processes. Particularly preferred test microorganisms include Bacillus, Clostridium, Neurospora, and Candida species of microorganisms. Spores from Bacillus and Clostridia species are the most commonly used to monitor sterilization processes utilizing saturated steam, dry heat, gamma irradiation, and ethylene oxide.

Particularly preferred microorganisms commonly used to monitor sterilization conditions include Geobacillus stearothermophilus and Bacillus atrophaeus. Geobacillus stearothermophilus is particularly useful to monitor sterilization under steam sterilization conditions and sterilization using oxidative sterilants. The enzyme alpha-D-glucosidase has been identified in spores of Geobacillus stearothermophilus, such as those commercially available as “ATCC 7953” from American Type Culture Collection, Rockville, Md. Bacillus atrophaeus is particularly useful to monitor conditions of gas and dry heat sterilization. The enzyme beta-D-glucosidase has been found in Bacillus atrophaeus (e.g., commercially available as “ATCC 9372” from American Type Culture Collection).

Where dual rapid-readout indicators are used, these microorganisms may serve as both the source of active enzyme in the rapid enzyme test and the test microorganism for the microorganism outgrowth test. Geobacillus stearothermophilus is particularly preferred for monitoring both steam and hydrogen peroxide plasma sterilization procedures. Bacillus atrophaeus is particularly preferred for monitoring ethylene oxide sterilization procedures and may be used to monitor hydrogen peroxide plasma sterilization procedures.

The present disclosure, although herein described primarily in terms of a single test microorganism species, should be understood to refer as well to the use is of a plurality of test microorganism species. For example, a single sterility indicator may contain two or more species of test microorganisms; one species being resistant to oxidative sterilant vapors and at least one species selected from the group consisting of a species being resistant to heat, a species being resistant to gaseous sterilizing media, and a species being resistant to radiation.

Enzyme substrates that are suitable for use in the biological sterilization indicators of the disclosure are described in U.S. Pat. No. 5,252,484 (Matner et al.) and U.S. Pat. No. 5,073,488 (Matner et al.). Chromogenic and fluorogenic substrates that react with enzymes to form detectable products, and that are suitable for use in the biological sterilization indicator of the disclosure, are well known in the art. These substrates may be classified in two groups based on the manner in which they create a visually detectable signal. The substrates in the first group react with enzymes to form enzyme-modified products that are themselves chromogenic or fluorescent. The substrates in the second group form enzyme-modified products that must react further with an additional compound to generate a color or fluorescent signal.

The present disclosure also provides methods of use of a self-contained biological sterilization indicator. In general, the present disclosure provides a method for testing the effectiveness of a sterilization procedure, the method comprising: providing a any embodiment of a self-contained biological sterilization indicator according to the present disclosure; subjecting the self-contained biological sterilization indicator comprising the test microorganism to a sterilization procedure; subsequent to sterilization, subjecting the self-contained biological sterilization indicator to a developing procedure to determine whether a detectable indication is present or absent; and correlating the presence of the detectable indication with failure of the sterilization procedure and the absence of the detectable indication with success of the sterilization procedure.

Using an exemplary spore outgrowth self-contained biological sterilization indicator, a method for testing the effectiveness of a sterilization procedure includes: providing a biological sterilization indicator comprising: an outer container having at least one liquid-impermeable wall and an interior volume; a sealed, openable, liquid-impermeable inner container enclosing a predetermined volume of an aqueous medium; a dry coating that comprises i) a plurality of viable test microorganisms useful to detect exposure to an oxidative sterilant and ii) an effective amount of a sterilant-resistance modulator; and a pathway that permits vapor communication between the interior volume and an atmosphere outside the outer container; wherein the inner container and the dry coating are disposed in the interior volume; wherein the modulator comprises an amino acid; wherein the effective amount causes an increase in sensitivity of the biological indicators to the oxidative sterilant relative to an otherwise-identical dry coating that lacks the effective amount. The method further comprises exposing the biological sterilization indicator a sterilization procedure; subsequent to exposing the biological sterilization indicator to the sterilization procedure, contacting the test microorganisms with a means for forming a detectable indication of the failure of a sterilization procedure; incubating the mixture of the test microorganisms with the means for forming a detectable indication under conditions that facilitate growth of the test microorganisms; observing a presence or absence of the detectable indication; and correlating the presence of the detectable indication with failure of the sterilization procedure or correlating the absence of the detectable indication with success of the sterilization procedure.

In a particularly preferred embodiment, the biological sterilization indicator is a dual rapid-readout indicator and the test microorganisms serve as both the source of active enzyme for the enzyme activity test and as the test microorganism for the microorganism outgrowth test. Suitable microorganisms include Geobacillus stearothermophilus and Bacillus atrophaeus. In the most preferred embodiment, Geobacillus stearothermophilus spores are used in the biological sterilization indicators.

Referring to FIGS. 1 and 2, in an exemplary use wherein the test microorganisms includes spores, the self-contained biological sterilization indicator 10 is placed into a sterilization chamber and exposed to a hydrogen peroxide vapor sterilization procedure. Sterilant enters the self-contained biological sterilization indicator 10 through vent 28 and closure member 22, and contacts the test microorganisms supported by the carrier 16. After the procedure is completed, the self-contained biological sterilization indicator 10 is removed from the sterilization chamber and the sides of the outer container 12 are compressed, breaking the frangible inner container 18 and releasing the nutrient medium that includes a pH indicator so that the medium may contact the test microorganisms supported by carrier 16. The self-contained biological sterilization indicator 10 is then incubated (e.g., at a suitable temperature (e.g., 55-60° C.) for a period of time sufficient for any surviving test microorganisms remaining in the indicator to grow and cause a color change in the pH indicator, which provides a detectable indication of the failure of the sterilization procedure. If the sterilization procedure is effective and all spores have been inactivated, then no color change is observed after the incubation period.

Referring to FIGS. 1 and 2, in an exemplary use wherein the test microorganisms produce an active enzyme, the self-contained biological sterilization indicator 10 is placed in the sterilization chamber and exposed to a hydrogen peroxide vapor sterilization procedure. Sterilant enters the indicator 10 through vent 28 and closure member 22, and contacts the source of test microorganisms supported by the carrier 16. After the procedure is completed, the self-contained biological sterilization indicator 10 is removed from the sterilization chamber and the sides of the outer container 12 are compressed, breaking the frangible inner container 18 and releasing the enzyme substrate so that it may contact the active enzyme produced by the test microorganisms. The self-contained biological sterilization indicator 10 is then incubated for a period of time sufficient for any active enzyme produced by the test microorganisms to react with the substrate and form a detectable product, which provides a detectable indication of the failure of the sterilization procedure. The detectable product may be detected by its characteristic fluorescence, luminescence, or its absorbance spectrum (e.g., color), for example. If the sterilization procedure is effective and all test microorganisms have been inactivated, then no detectable signal is generated observable after the incubation period.

Another exemplary self-contained biological sterilization indicator of the present disclosure is shown in FIGS. 3-4. The self-contained biological sterilization indicator 200 includes a housing 202, which contains a first portion 204 (e.g., a hollow tube) and a second portion 206 (e.g., a cap) that are coupled together to provide a self-contained biological sterilization indicator. The cap may be a molded polypropylene with general dimensions of approximately 21 mm long by 14 mm in diameter. The first portion 204 (hollow tube) may be a molded polycarbonate piece, with the general dimensions of about 52 mm long and 12 mm in diameter at top, with the shape shown in FIGS. 3-4. The total volume of the first portion 204 (e.g., a hollow tube) is approximately 3 mL, for example.

The housing 202 can be defined by at least one liquid impermeable wall, such as a wall 208 of the first portion 204 and/or a wall 210 of the second portion 206. It should be understood that a one-part unitary housing 202 may also be employed or that the first and second portions 204 and 206 can take on other shapes, dimensions, or relative structures without departing from the spirit and scope of the present disclosure. Suitable materials for the housing 202 (e.g., the walls 208 and 210) can include, but are not limited to, a glass, a metal (e.g., foil), a polymer (e.g., polycarbonate (PC), polypropylene (PP), polyphenylene (PPE), polythyene, polystyrene (PS), polyester (e.g., polyethylene terephthalate (PET)), polymethyl methacrylate (PMMA or acrylic), acrylonitrile butadiene styrene (ABS), cyclo olefin polymer (COP), cyclo olefin copolymer (COC), polysulfone (PSU), polyethersulfone (PES), polyetherimide (PEI), polybutyleneterephthalate (PBT)), a ceramic, a porcelain, or combinations thereof.

In any embodiment, the second portion (cap) 206 of the housing 202 may include one or more aperture or opening 207, which provide fluid communication between the interior of the housing 202 (e.g., the reservoir 203) and ambience. In any embodiment, the second portion 206 of the housing 202 may include a plurality (e.g., six) of openings 207. A filter paper material (not shown) which acts as a microbial barrier; is positioned in the sterilant path over the openings 207 and held in place with a pressure sensitive adhesive backed paper label. The filter paper material is the same material present in the cap of currently-available 3M ATTEST 1291 Rapid Readout Biological indicators for Steam Sterilizers (available from 3M Company, St. Paul, Minn.).

The self-contained biological sterilization indicator 200 further includes a frangible container 220 that contains a liquid nutrient medium 222. The frangible container 220 is made of borosilicate glass and contains the nutrient medium which facilitates growth of spores, for example. The medium consists of a modified Tryptic Soy Broth (TSB) containing a pH indicator, bromocresol purple, and a fluorogenic enzyme substrate, 4-Methylumbelliferyl-alpha-D-glucoside. The ampoule is approximately 40 mm long by about 4 mm in diameter, for example, and holds approximately 500 μL of liquid nutrient medium, for example. An example of a suitable liquid nutrient medium 222 is the medium used in the product currently-available from 3M Company as 3M ATTEST 1292 Rapid Readout Biological indicators for Steam Sterilizers.

The liquid medium container 220 may be held in place within the self-contained biological sterilization indicator 200 by an insert 230. The insert (also called a breaker) 230 functions to hold the container 220 in place as well as to facilitate the controlled breakage of the container 220. Controlled breakage occurs during the activation step of the self-contained biological sterilization indicator, when the second portion (cap) 206 is urged downward (i.e., toward the first portion 204 of the housing) to break the container 220. The insert 230 may be a molded polycarbonate structure with approximate dimensions of 22 mm long by 9 mm wide, for example.

The second portion 206 has a seal positioned to contact the first end 201 of the first portion 204, at the open upper end of the first portion 204 to close or seal (e.g., hermetically seal) the self-contained biological sterilization indicator 200 after activation.

The self-contained biological sterilization indicator 200 further includes a dry coating 292 that comprises suitable sterilant-resistant spores such as G. stearothermophilus spores (ATCC 7953) and an effective amount of a sterilant resistance modulator according to the present disclosure, the dry coating 292 positioned in fluid communication with the first portion 204. The dry coating 392 of the illustrated embodiment of FIGS. 3-4 is deposited on a carrier 116.

The housing 202 includes a lower portion 214 (that at least partially defines a first chamber 209) and an upper portion 216 (that at least partially defines a second chamber 211), which are partially separated by an inner partial wall or ledge 218, in which is formed an opening 217 that provides fluid communication between the first chamber 209 and the second chamber 211. The second chamber 211 is adapted to house the carrier 116. The first chamber 209 is adapted to house the frangible container 220, particularly before activation. The wall 218 is angled or slanted, at a non-zero and non-right angle with respect to the longitudinal direction D_{L} of the housing 202.

The second chamber 211, which can also be referred to as the “test microorganism growth chamber” or “detection chamber,” includes a volume to be interrogated for test microorganism viability to determine the efficacy of a sterilization process.

The liquid medium container 220 is positioned and held in place by insert 230 in the first chamber 209. The dry coating 292 comprising the test microorganism and the sterilant resistance modulator is positioned on the carrier 116 and housed in the second chamber 211 and in fluid communication with ambience during sterilization. The sterilant moves into the second chamber 211 (e.g., via the first chamber 209) during sterilization. After being exposed to a sterilization process, the self-contained biological sterilization indicator is intentionally activated and the liquid medium 222 moves into the second chamber 211 (e.g., from the first chamber 209) when the container 220 is fractured and the liquid medium 222 is released into the interior of the housing 202.

The first chamber 209 has a volume of about 2800 microliters (empty of all internal components), for example. The cross-sectional area of the first chamber 209, immediately above the wall 218 is approximately 50 mm², for example. The second chamber 211 has a volume of about 210 microliters, for example. The cross-sectional area of the second chamber 211, immediately below the wall 218, is approximately 20 mm², for example.

The housing 202 is tapered (see, e.g., the tapered portion 246) so that the cross-sectional area in the housing 202 generally decreased along the longitudinal direction D_{L} from the first end 201 of first portion 204 to the closed end 205 of the housing 202.

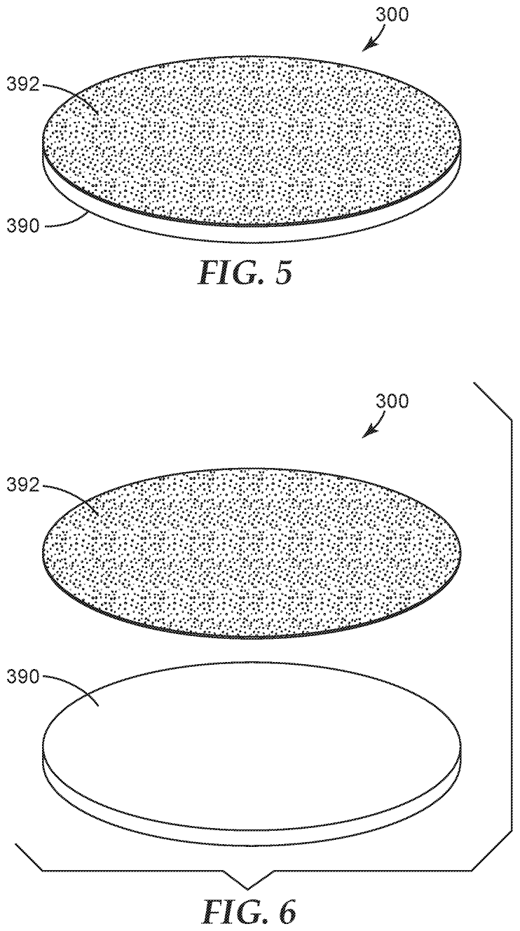

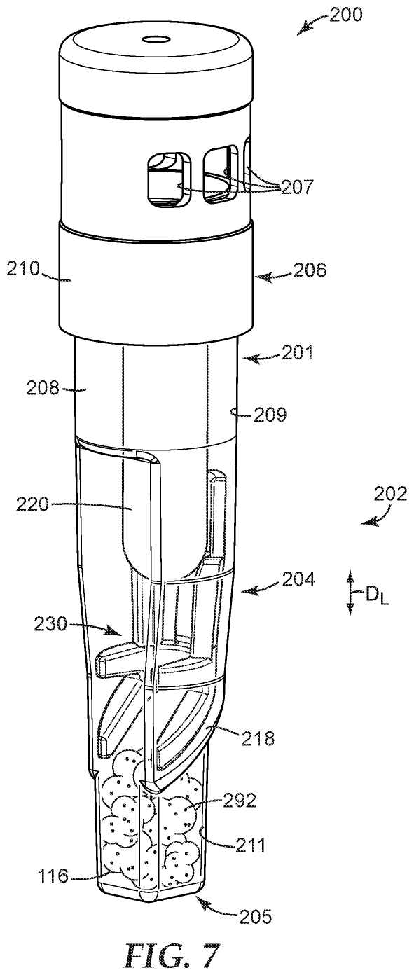

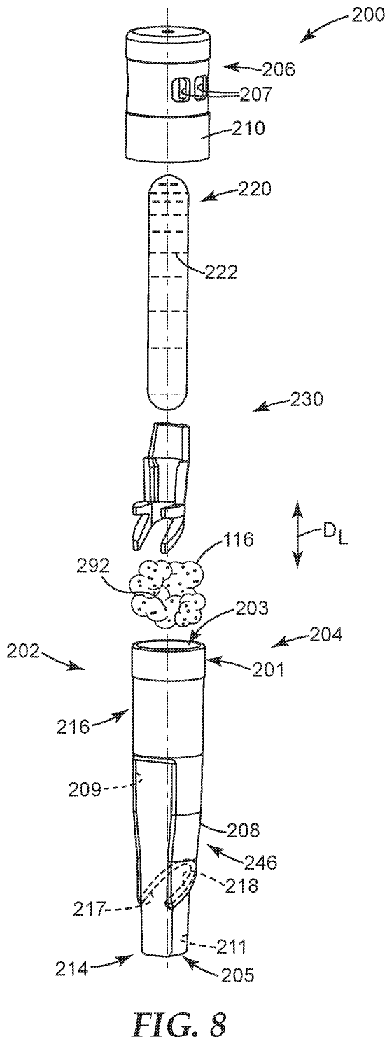

In another aspect, the present disclosure provides a biological sterilization indicator. FIGS. 4 and 5 show two views of one embodiment of a biological sterilization indicator 300 according to the present disclosure. The biological sterilization indicator 300 comprises a carrier 390 with a dry coating 392 disposed thereon.

The carrier 390 can be made of a material that is hydrophobic or hydrophilic. Such materials can be inorganic, organic, or combinations thereof. Carriers that include (or are prepared from) hydrophobic materials can be used with any indicator, whereas carriers that include (or are prepared from) hydrophilic materials are preferably used to monitor a sterilization procedure that uses hydrogen peroxide vapor phase. Examples of suitable hydrophobic materials include polypropylene, polyethylene, PET, polyurethane, nylon, polymer blends containing one or more of these polymers (e.g., with other hydrophobic polymers), or combinations thereof. Examples of suitable hydrophilic materials include glass. Other suitable materials for use as a carrier include glass fibers and metals (e.g., a stainless steel coupon) that do not substantially react with the sterilant.

The carrier 390 may be provided in any of a variety of shapes and sizes. Carriers made from relatively thin, flexible sheet-like materials (e.g., polymeric films) are particularly suitable although metal, glass, or ceramic coupons may also be used. In any embodiment, the carrier 390 may be substantially transmissive to electromagnetic radiation of the visible and/or ultraviolet wavelengths (e.g., the carrier may be transparent or translucent). Alternatively, or additionally, portions (or all) of the carrier may be substantially nontransmissive to electromagnetic radiation of the visible and/or ultraviolet wavelengths (e.g., opaque). In any embodiment, the carrier may substantially reflect electromagnetic radiation of the visible and/or ultraviolet wavelengths.

Returning back to FIGS. 5 and 6, a coating 392 is disposed on the carrier 390. The coating 392 is dry (i.e., substantially water-free, as defined herein). In any embodiment, the coating 392 comprises a plurality of test microorganisms. The test microorganisms are microorganisms commonly used to monitor the effectiveness of a sterilization procedure. In any embodiment, the test microorganisms may produce (e.g., during sporulation and/or growth) an active enzyme whose activity is correlated with the survival of the test microorganism after it has been exposed to a sterilant in a sterilization process.

The test microorganisms supported by the carrier 390 are selected such that they are inactivated (e.g., killed) by a sterilization procedure that is lethal to the test microorganism. In contrast, wherein the test microorganisms are not inactivated by a sterilization procedure that is sublethal to the test microorganism, the test microorganisms provide a detectable indication (e.g., growth) as a result of an ineffective sterilization procedure. Preferably, for enzyme-based detection, the test microorganisms produce an active enzyme, the enzyme having an enzyme activity that is correlated with the survival of at least one test microorganism. Thus, the active enzyme is inactivated by a sterilization procedure that is lethal to the test microorganism, but the active enzyme is not inactivated by a sterilization procedure that is sublethal to the test microorganism.

Suitable test microorganisms for use in a biological sterilization indicator of the present disclosure include species of microorganisms from the genera Bacillus, Geobacillus, Clostridium, Neurospora, and Candida, for example. Organisms from the aforementioned species are known to produce active enzymes that that can be used to detect survival of the test microorganisms after being exposed to a sterilant in a sterilization process.

In any embodiment, the dry coating 392 comprises a predetermined quantity of viable test microorganisms. The quantity is typically quantified as colony-forming units (CFUs) using plate count methods that are well-known in the art. In any embodiment, the dry coating comprises about 10² viable test microorganisms to about 10⁹ viable test microorganisms. In any embodiment, the dry coating comprises about 10² viable test microorganisms to about 10⁸ viable test microorganisms. In any embodiment, the dry coating comprises about 10⁴ viable test microorganisms to about 10⁵ viable test microorganisms. In any embodiment, the dry coating comprises about 10⁵ viable test microorganisms to about 10′ viable test microorganisms.

The dry coating 392 further comprises an effective amount of a sterilant resistance modulator. The effective amount of the sterilant resistance modulator in the dry coating 392 causes an increase in sensitivity of the biological indicators to an oxidative sterilant (e.g., vapor phase hydrogen peroxide), relative to an otherwise-identical dry coating that lacks the effective amount. The sterilant resistance modulator comprises an amino acid. In any embodiment, the amino acid is selected from a group consisting of L-homocysteine, L-arginine, L-histidine, or a combination of any two or more of the foregoing amino acids.

In any embodiment, the dry coating 392 comprises a predetermined quantity of the sterilant resistance modulator. The predetermined quantity is an amount effective to increase the sensitivity of the biological indicators to the oxidative sterilant relative to an otherwise-identical dry coating that lacks the effective amount. The sensitivity of the biological indicators to the oxidative sterilant, and the effect of the modulator on the sensitivity of the biological indicators to the sterilant, can be determined easily as described in the Examples. As evidenced by the examples, the sensitivity to the sterilant can be measured by growth of the test microorganisms after exposure to the sterilant and/or by detecting an enzyme activity associated with the test microorganisms after the microorganisms have been exposed to the sterilant.

When the dry coating 392 comprises about 10⁶ test microorganisms, an effective amount of sterilant resistance modulator is about 2 to about 20 micrograms (e.g., about 11.5 to about 150 nmoles). The effective amount of sterilant resistance modulator may also be expressed in terms of mass of sterilant resistance modulator per viable test microorganism. In any embodiment the effective amount of sterilant resistance modulator is about 0.1 femtomole/viable test microorganism to about 1.5 femtomoles/viable test microorganism.

The present disclosure also provides methods of use of a biological sterilization indicator. In general, the present disclosure provides a method for testing the effectiveness of a sterilization procedure, the method comprising: providing a any embodiment of a biological sterilization indicator according to the present disclosure; subjecting the biological sterilization indicator comprising the test microorganism to a sterilization procedure; subsequent to sterilization, subjecting the biological sterilization indicator to a developing procedure to determine whether a detectable indication is present or absent; and correlating the presence of the detectable indication with failure of the sterilization procedure and the absence of the detectable indication with success of the sterilization procedure.

Using an exemplary spore outgrowth biological sterilization indicator, a method for testing the effectiveness of a sterilization procedure includes: providing any embodiment of a biological sterilization indicator comprising a carrier with a dry coating disposed thereon, the dry coating comprising i) a plurality of viable test microorganisms useful to detect exposure to an oxidative sterilant and ii) an effective amount of a sterilant-resistance modulator as described herein. The method further comprises exposing the biological sterilization indicator a sterilization procedure; subsequent to exposing the biological sterilization indicator to the sterilization procedure, contacting the test microorganisms with a means for forming a detectable indication of the failure of a sterilization procedure; incubating the mixture of the test microorganisms with the means for forming a detectable indication under conditions that facilitate growth of the test microorganisms; observing a presence or absence of the detectable indication; and correlating the presence of the detectable indication with failure of the sterilization procedure or correlating the absence of the detectable indication with success of the sterilization procedure.

In any embodiment, contacting the test microorganisms with a means for forming a detectable indication of the failure of a sterilization procedure can comprise contacting the biological sterilization indicator with a nutrient medium (i.e., by placing the carrier into a tube of broth medium), for example. If a test microorganism on the carrier survived the sterilization procedure, it can grow in the nutrient medium, wherein the growth can be detected by turbidity and/or by a detectable pH change. Alternatively, or additionally, the carrier may be placed in a medium comprising an enzyme substrate that, upon reaction with an enzyme produced by a surviving test microorganism, can produce a detectable product, as described hereinabove.

Any of the biological sterilization indicators of the present disclosure may be used as part of a test pack. In one embodiment of the disclosure, a non-challenge test pack of the disclosure provides no additional resistance to the sterilization procedure above the resistance of the biological sterilization indicator alone. The non-challenge test pack provides an advantage over the use of the indicator without a test pack in that it securely holds the biological sterilization indicator in a single position during the sterilization procedure. The non-challenge test pack thus alleviates a problem that occurs when biological sterilization indicators, which are typically small and prone to roll about, become displaced or misplaced in a load of materials during a sterilization procedure.

An alternative test pack, referred to as a lumen-challenge test pack, which provides additional resistance to a biological sterilization indicator that is equivalent to the resistance the indicator would experience if placed within a lumen having a defined cross-sectional area and length. Lumen-challenge test packs provide an accurate method of determining whether a sterilization procedure would be effective in killing microorganisms that may be located deep within the interior of a tube-like instrument. Exemplary non-challenge and lumen-challenge sterilization test packs are described in U.S. Pat. No. 6,897,059 (Foltz et al.).

Certain embodiments of the methods, compositions, articles, and kits of the present disclosure are set forth in the following list of embodiments.

### EXEMPLARY EMBODIMENTS

Embodiment A is a self-contained biological sterilization indicator, comprising:

an outer container having liquid-impermeable walls and an interior volume;

a sealed, openable, liquid-impermeable inner container enclosing a predetermined volume of an aqueous medium;

a dry coating that comprises i) a plurality of viable test microorganisms useful to detect exposure to an oxidative sterilant and ii) an effective amount of a sterilant-resistance modulator; and

a pathway that permits vapor communication between the interior volume and an atmosphere outside the outer container;

wherein the inner container and the dry coating are disposed in the interior volume;

wherein the modulator comprises an amino acid;

wherein the effective amount causes an increase in sensitivity of the biological indicators to the oxidative sterilant relative to an otherwise-identical dry coating that lacks the effective amount.

Embodiment B is the self-contained biological sterilization indicator of Embodiment A, wherein the outer container comprises at least one wall, wherein at least a portion of the dry coating is disposed in the interior volume on the at least one wall.

Embodiment C is the self contained biological sterilization indicator of Embodiment A, further comprising a carrier, wherein at least a portion of the dry coating is disposed on the carrier.

Embodiment D is the self-contained biological sterilization indicator of Embodiment C; wherein the carrier comprises glass, metal a non-cellulosic polymer, or combinations thereof.

Embodiment E is the self-contained biological sterilization indicator of any one of the preceding Embodiments, wherein the dry coating is disposed in vapor communication with the atmosphere outside the container.

Embodiment F is the self-contained biological sterilization indicator of any one of the preceding Embodiments, further comprising a means for forming a detectable indication of the failure of a sterilization procedure.

Embodiment G is the self-contained biological sterilization indicator of any one of the preceding claims, wherein the modulator is selected from the group consisting of L-homocysteine, L-arginine, and L-histidine.

Embodiment H is the self-contained biological sterilization indicator of any one of the preceding Embodiments, wherein the dry coating comprises about 10³ viable test microorganisms to about 10⁸ viable test microorganisms.

Embodiment I is the self-contained biological sterilization indicator of Embodiment G, wherein the dry coating comprises about 10⁴ viable test microorganisms to about 10⁷ viable test microorganisms.

Embodiment J is the self-contained biological sterilization indicator of any one of the preceding Embodiments, wherein the effective amount is about 2 micrograms to about 20 micrograms.

Embodiment K is the self-contained biological sterilization indicator of any one of the preceding Embodiments, wherein the effective amount is about 11.5 nmoles to about 150 nmoles.

Embodiment L is the self-contained biological sterilization indicator of any one of the preceding Embodiments, wherein the effective amount is about 0.02 nanograms/viable test microorganism to about 0.2 nanograms/viable test microorganism.

Embodiment M is the self-contained biological sterilization indicator of any one of the preceding Embodiments, wherein the effective amount is about 0.1 femtomole/viable test microorganism to about 1.5 femtomoles/viable test microorganism.

Embodiment N is the self-contained biological sterilization indicator of any one of the preceding Embodiments, wherein the test microorganism is a spore.

Embodiment O is the self-contained biological sterilization indicator of Embodiment N, wherein the spore is a Geobacillus stearothermophilus spore.

Embodiment P is the self-contained biological sterilization indicator of any one of the preceding Embodiments, wherein the sterilant resistance modulator modulates resistance of the test organism to an oxidative sterilant or disinfectant comprising hydrogen peroxide, peracetic acid, ozone, or chlorine dioxide.

Embodiment Q is the self-contained biological sterilization indicator of any one of the preceding Embodiments, wherein the pathway is configured to hinder passage of a microorganism through the pathway.

Embodiment R is the self-contained biological sterilization indicator of any one of the preceding Embodiments, wherein the pathway is configured to hinder passage of a microorganism through the pathway.

Embodiment S is a biological sterilization indicator, comprising:

a carrier; and

a dry coating disposed thereon;

wherein the dry coating comprises i) a plurality of viable test microorganisms useful to detect exposure to an oxidative sterilant and ii) an effective amount of a sterilant-resistance modulator;

wherein the effective amount causes an increase in sensitivity of the biological indicators to the oxidative sterilant relative to an otherwise-identical dry coating that lacks the effective amount.

Embodiment T is the biological sterilization indicator of Embodiment S, wherein the modulator is selected from the group consisting of L-homocysteine, L-arginine, L-histidine and mixtures thereof.

Embodiment U is the biological sterilization indicator of Embodiment S or Embodiment T, wherein the effective amount is about 2 micrograms to about 20 micrograms.

Embodiment V is the biological sterilization indicator of any one of Embodiments S through U, wherein the effective amount is about 11.5 nmoles to about 150 nmoles.

Embodiment W is the biological sterilization indicator of any one of Embodiments S through V, wherein the effective amount is about 0.02 nanograms/viable test microorganism to about 0.2 nanograms/viable test microorganism.

Embodiment X is the self-contained biological sterilization indicator of any one of Embodiments S through W, wherein the effective amount is about 0.1 femtomole/viable test microorganism to about 1.5 femtomoles/viable test microorganism.

Embodiment Y is the biological sterilization indicator of any one of Embodiments S through X, wherein the test microorganism is a spore.

Embodiment Z is the biological sterilization indicator of Embodiment Y, wherein the spore is a Geobacillus stearothermophilus spore.

Embodiment AA is the biological sterilization indicator of any one of Embodiments S through Z, wherein the sterilant resistance modulator modulates resistance of the test organism to an oxidative sterilant or disinfectant comprising hydrogen peroxide, peracetic acid, ozone, chlorine dioxide, and a combination of any two or more of the foregoing oxidative sterilants.

Embodiment AB is the biological sterilization indicator of any one of Embodiments S through AA; wherein the carrier comprises glass, metal a non-cellulosic polymer, or combinations thereof.

Embodiment AC is a method, comprising:

providing the self-contained biological sterilization indicator of any one of Embodiments A through R;

exposing the self-contained biological sterilization indicator to a sterilant in a sterilization process, wherein the sterilant is an oxidative sterilant; and

detecting an indication whether at least one microorganism of the plurality of test microorganisms survived the sterilization process.

Embodiment AD is a method, comprising:

providing the biological sterilization indicator of any one of Embodiments S through AB;

exposing the self-contained biological sterilization indicator or the biological sterilization indicator to a sterilant in a sterilization process, wherein the sterilant is an oxidative sterilant; and

detecting an indication whether at least one microorganism of the plurality of test microorganisms survived the sterilization process.

Embodiment AE is the method of Embodiment AC or Embodiment AD, wherein detecting an indication whether at least one of the plurality of test microorganisms survived the sterilization process comprises detecting growth of the test microorganism.

Embodiment AF is the method of Embodiment AC or Embodiment AD, wherein detecting an indication whether at least one of the plurality of test microorganisms survived the sterilization process comprises detecting a predetermined enzyme activity associated with the test microorganism.

Objects and advantages of this invention are further illustrated by the following examples, but the particular materials and amounts thereof recited in these examples, as well as other conditions and details, should not be construed to unduly limit this invention.

EXAMPLES

Materials

TABLE 1
List of materials used in the Examples.

| Chemicals | Manufacturer | Location |
|---|---|---|
| L-histidine | J. T. Baker | Austin, TX |
| monohydrochloride | | |
| L-homocysteine | P212121 | Toledo, OH |
| Proline | Spectrum Chemical | New Brunswick, NJ |
| L-arginine | Spectrum Chemical | New Brunswick, NJ |
| hydrochloride | | |

Sterilizer System and Sterilization Process Parameters.

The self-contained biological indicators (BIs) described in the Examples below, were exposed to sterilization processes using hydrogen peroxide sterilization with a STERRAD® NX® sterilizer (available from Advanced Sterilization Products (ASP) of Irvine, Calif.). The exposure time was either standard or varied as described in more detail in the Examples below. A detailed description of each sterilization process is shown in Table 2.

TABLE 2
Sterilization process parameters.

| | | Process I | Process II |
|---|---|---|---|
| Sterilization | Controlled | (Standard | (Half |
| Process Stage | parameter | Cycle) | Cycle) |
| 1) Delivery | Volume of 59% H₂O₂ | 1.8 mL | 1.8 mL |
| | solution admitted | | |
| | into the sterilizer | | |
| 2) Exposure | Exposure to H₂O₂ | 3 minutes | 3 minutes |

| | | |
|---|---|---|
| Approx. cycle duration | 28 minutes | 14 minutes |
| (empty chamber) | | |

### Example 1

Modulation by L-Homocysteine of Spore Resistance to a Hydrogen Peroxide Sterilization Processes.

Tub-shaped spore carriers (polypropylene), similar to those described in International Publication No. WO2014/189716 with the exception that they were not coated with colloidal nanoparticles, were used. Liquid-cultured Geobacillus stearothermophilus spores (>10⁶), were suspended in sterile water. Approximately 10 μL of the spore suspension was deposited onto the spore carriers and dried at room temperature. Aliquots (0 μL, 5 μL, or 10 μL, respectively) of a 0.5M sterile solution of L-homocysteine were deposited into the dried, spore-coated tub-shaped spore carrier. The homocysteine-coated tubs were dried at room temperature. Thus, the final dried coating on the carriers included at least 10⁶ spores with 0, 2.5 or 5 micromoles of L-homocysteine, respectively.

Self-contained biological indicators were assembled similarly to those shown in FIGS. 1 and 2 with the exception that the tub-shaped spore carrier was placed into the bottom of the outer container 12 before the inner container 18 was placed into the outer container and the vented cap 26 had a single circular opening (2.3 mm dia.) located on the top (flat) surface of the cap, rather than the rectangular opening 28 shown on the curved surface of the cap 26 in

FIG. 1. The outer container 12 was obtained from 3M ATTEST 1292 Rapid Biological Indicators (available from 3M Company; St. Paul, Minn.). The cap was obtained from 3M ATTEST 1261 Biological Indicators (3M Company). The nutrient medium was from 3M ATTEST 1292 ampoules with added methionine (5 g/L). The medium was sterilized in sealed glass ampoules, which were placed into the outer container as shown in FIG. 2. Five individual self-contained biological indicators were exposed to different concentrations (listed in Table 3) of hydrogen peroxide for 2 minutes in a STERRAD NX sterilizer. After exposing them to the sterilization process, all of the self-contained biological indicators were activated by crushing the ampoule inside the outer container. The self-contained biological indicators were incubated at 56° C. up to seven days and the pH indicator in the medium was observed to determine whether any of the spores survived the sterilization process. The results are shown in Table 3. All positive controls showed an indication of growth (data not shown).

TABLE 3
Survival of spores in Sterilant Resistance Modulator-Treated
Self-contained Biological Indicators after exposure to H₂O₂ Sterilant.
The numbers in the table show the percentage of self-contained
biological indicators that showed observable growth (i.e., pH
indicator change) after exposure to the sterilization process.

| Volume of 59% H₂O₂ | 0.1 ml | 0.2 ml | 0.4 ml | 0.6 ml | 0.8 ml |
|---|---|---|---|---|---|
| mg × Sec/L of H₂O₂ | 94 | 222 | 458 | 689 | 842 |
| spores + 5.0 micromoles of | 100% | 40% | 0% | 0% | 0% |
| homocysteine | | | | | |
| spores + 2.5 micromoles of | 100% | 80% | 60% | 60% | 20% |
| homocysteine | | | | | |
| spores + no additional | 100% | 100% | 100% | 100% | 100% |
| homocysteine | | | | | |

All unexposed self-contained BI positive controls were growth-positive within 24 hours of incubation. Self-contained biological indicators prepared with 2.5 micromoles of L-homocysteine showed a decrease in resistance of spores compared to those with 0 micromoles of L-homocysteine, as seen by the decreased number of growth-positive BI with 0.6 ml and 0.8 ml of 59% H₂O₂ respectively. The amount of 5.0 micromoles of L-homocysteine reduced the resistance of spores significantly as seen by all kill with 0.4 ml of 59% H₂O₂.

### Example 2

### Modulation by L-Arginine and L-Histidine of Spore Resistance to a Hydrogen Peroxide Sterilization Processes.

Preparation of Self-contained Biological Indicator.

PET film (.09 mm thick) was coated with colloidal silica as described in International Publication No. WO2014/189716, which is incorporated herein by reference in its entirety. Liquid cultured spore crops of Geobacillus stearothermophilus were washed in sterile distilled water and were suspended in sterile water to a concentration such that a 1:1000 dilution (in water) of the spore suspension had an optical transmittance (625 nm wavelength) of 37%. Separate aqueous spore coating solutions were prepared as described in Example 1 with the amino acids shown in table 4 present in the coating solutions. Two microliter aliquots (containing at least 10⁶ spores) of the respective spore coating solutions were deposited onto the silica-coated PET film, forming a series of spaced-apart spots on the film. The spore coated films were dried in a 60° C. incubator for 12 minutes. Circular discs (hereinafter, “coated carriers”) of the PET film, each disc including one of the dried spots, were punched out and were used to assemble self-contained biological indicators (BI) similar to that shown in FIG. 1 of International Publication No. WO2014/189716. The coated carriers described above were placed into the “spore carrier 135” shown in FIG. 1 of International Publication No. WO2014/189716. Each self-contained BI comprised a polycarbonate outer container, a breaker, a cap, and a cap filter. Disposed inside the outer container were a coated carrier along with an ampoule media containing the nutrient medium from 3M ATTEST 1292 Rapid Readout Biological Indicators plus 5g/L methionine (as described in Example 1).

Hydrogen Peroxide Sterilization

The self-contained biological indicators with different concentrations of additives (Table 4) were exposed to hydrogen peroxide sterilization in STERRAD NX sterilizer. The sterilization processes were run by manually injecting 1.0 ml of 59% hydrogen peroxide per load, unless specified otherwise. The exposure time was varied to determine the resistance profile of self-contained BIs with different additive formulations. The BIs were activated by pressing down the cap to break the media ampoule. The activated BIs were placed in a bench-top fluorimeter (3M Company, St. Paul, Minn.) to detect fluorescence.

Monitoring the Self-Contained Biological Indicators for Surviving Spores

A self-contained BI that is not completely inactivated (i.e., not all of the spores are killed by the sterilization process) will resume cellular functions upon activation of the BI. The production of glucosidase enzyme by surviving spores is an indication that at least one of the spores have not been inactivated (killed) by the sterilization process. The glucosidase enzyme cleaves the 4-methylumbelliferyl-glucoside (MUG) releasing the fluorescent methyl umbelliferone, which is detected using a bench-top fluorimeter. Growth and proliferation of spores in the self-contained biological indicator can also be detected by a pH change, which may be detected by a color change of the pH indicator in the growth medium.

Effect of different additives on the resistance of biological indicators to hydrogen peroxide sterilization.

Self-contained biological indicators were prepared (with various additives listed in table 4) as described above in this Example. For each additive tested, a lower range and a higher range was used as a preliminary screen to identify additives modulated the resistance of the spores to the sterilant. Five self-contained BIs representing each condition were exposed to hydrogen peroxide sterilization in STERRAD NX sterilizer. The sterilization processes used a constant injection volume (1.0 ml of 59% hydrogen peroxide) and various times (e.g., 20 seconds to 7 minutes) of exposure to the hydrogen peroxide. The self-contained BIs were activated after exposure to the sterilization process and were monitored for fluorescence using a bench-top fluorimeter) and pH based color change (visually). The fluorescence and growth readout results are shown in Table 4.

### Comparative Example 1

### Lack of Modulation by L-Proline of Spore Resistance to a Hydrogen Peroxide Sterilization Processes.

Self-contained biological indicators with L-proline were prepared, processed in a sterilizer, and analyzed as described in Example 2. The self-contained BIs were activated after exposure to the sterilization process and were monitored for fluorescence using a bench-top fluorimeter and pH based color change (visually). The fluorescence and growth readout results are shown in Table 4.

TABLE 4
Resistance of Self-contained Biological Indicators to hydrogen peroxide sterilization. The values listed in parentheses
in Table 4 are the concentration (weight percent) of the sterilant resistance modulator in the aqueous solution used to
coat the spores on the carrier. The percentages listed in the table refer to the percentage of the self-contained biological
indicators that tested positive for glucosidase enzyme activity (“ENZ+”) and the percentage of the self-contained
biological indicators that tested positive for growth (“GRO+”), respectively.

| Resistance | H₂O₂ Exposure Period | | | | | | | | | |
|---|---|---|---|---|---|---|---|---|---|---|
| Modulator | 30 sec. | | 60 sec. | | 90 sec. | | 3 min. | | 7 min. | |
| (Concentration) | ENZ+ | GRO+ | ENZ+ | GRO+ | ENZ+ | GRO+ | ENZ+ | GRO+ | ENZ+ | GRO+ |
| L-arginine (0.1) | 80% | 40% | 0% | 0% | 0% | 0% | 0% | 0% | 0% | 0% |
| L-arginine (0.01) | 80% | 100% | 60% | 0% | 0% | 0% | 0% | 0% | 0% | 0% |
| L-histidine (0.1) | 100% | 0% | 10% | 0% | 0% | 0% | 0% | 0% | 0% | 0% |
| L-histidine (0.01) | 100% | 80% | 80% | 0% | 0% | 0% | 0% | 0% | 0% | 0% |
| L-proline (0.1) | 100% | 100% | 100% | 100% | 100% | 100% | 0% | 0% | 0% | 0% |
| L-proline (0.01) | 100% | 100% | 100% | 100% | 100% | 100% | 0% | 0% | 0% | 0% |
| No additives (0) | 100% | 100% | 100% | 100% | 100% | 100% | 10% | 10% | 0% | 0% |

The data indicate that the presence of L-arginine and L-histidine in the spore coating solution resulted in an increased sensitivity (decreased resistance), relative to the control with no additives, of the biological indicator to the effects of the sterilization process. In contrast, the presence of L-proline did not result in an increased sensitivity, relative to the control with no additives, of the biological indicators to the effects of the sterilization process.

### Example 3

### Modulation by L-Arginine and L-Histidine of Spore Resistance to a Hydrogen Peroxide Sterilization Processes.

Self-contained biological indicators (with various additives listed in table 5) were prepared as described in Example 2. The self-contained biological indicators were exposed to hydrogen peroxide in a STERRAD NX sterilizer as described in Example 2. The self-contained BIs were activated after exposure to the sterilization process and were monitored for fluorescence using a bench-top fluorimeter and pH based color change (visually). The fluorescence and growth readout results are shown in Table 5.

TABLE 5
Resistance of Self-contained Biological Indicators to hydrogen peroxide sterilization. The values listed in parentheses
in Table 5 are the concentration (weight percent) of the sterilant resistance modulator in the aqueous solution used to
coat the spores on the carrier. The percentages listed in the table refer to the percentage of the self-contained biological
indicators that tested positive for glucosidase enzyme activity (“ENZ+”) and the percentage of the self-contained
biological indicators that tested positive for growth (“GRO+”), respectively.

| Resistance | H₂O₂ Exposure Period | | | | | | | | | | | |
|---|---|---|---|---|---|---|---|---|---|---|---|---|
| Modulator | 20 sec. | | 30 sec. | | 60 sec. | | 90 sec. | | 3 min. | | 7 min. | |
| (Concentration) | ENZ+ | GRO+ | ENZ+ | GRO+ | ENZ+ | GRO+ | ENZ+ | GRO+ | ENZ+ | GRO+ | ENZ+ | GRO+ |
| Histidine (0.8) | 100% | 33% | 100% | 0% | 0% | 0% | 67% | 0% | 0% | 0% | 0% | 0% |
| Histidine (0.4) | 100% | 100% | 100% | 0% | 0% | 0% | 33% | 0% | 0% | 0% | 0% | 0% |
| Histidine (0.2) | 100% | 100% | 100% | 0% | 33% | 0% | 67% | 0% | 0% | 0% | 0% | 0% |
| Histidine (0.1) | 100% | 100% | 100% | 33% | 67% | 0% | 67% | 0% | 0% | 0% | 0% | 0% |
| Histidine (0.05) | 100% | 100% | 100% | 100% | 0% | 0% | 0% | 0% | 0% | 0% | 0% | 0% |
| Arginine (0.8) | 100% | 33% | 67% | 0% | 0% | 0% | 0% | 0% | 0% | 0% | 0% | 0% |
| Arginine (0.4) | 100% | 67% | 100% | 33% | 0% | 0% | 0% | 0% | 0% | 0% | 0% | 0% |
| Arginine (0.2) | 100% | 100% | 100% | 100% | 33% | 0% | 0% | 0% | 0% | 0% | 0% | 0% |
| Arginine (0.1) | 100% | 100% | 100% | 100% | 33% | 0% | 0% | 0% | 0% | 0% | 0% | 0% |
| Arginine (0.05) | 100% | 100% | 100% | 100% | 67% | 0% | 0% | 0% | 0% | 0% | 0% | 0% |
| No additives (0) | 100% | 100% | 100% | 100% | 100% | 100% | 100% | 100% | 0% | 0% | 0% | 0% |

The data indicate that the presence of L-arginine and L-histidine in the spore coating solution resulted in an increased sensitivity, relative to the control with no additives, of the spores to the damaging/lethal effects of the sterilization process.

The complete disclosure of all patents, patent applications, and publications, and electronically available material cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

Various modifications may be made without departing from the spirit and scope of the invention. These and other embodiments are within the scope of the following claims.

1. A self-contained biological sterilization indicator, comprising:
an outer container having at least one liquid-impermeable wall and an interior volume;
a sealed, openable, liquid-impermeable inner container enclosing a predetermined volume of an aqueous medium;
a dry coating that comprises i) a plurality of viable Geobacillus stearothermophilus spores suitable for detecting exposure to a hydrogen peroxide sterilant in a sterilization process and ii) an effective amount of a sterilant-resistance modulator; and
a pathway that permits vapor communication between the interior volume and an atmosphere outside the outer container;
wherein the inner container and the dry coating are disposed in the interior volume;
wherein the modulator is selected from the group consisting of L-homocysteine, L-arginine, and L-histidine;
wherein the effective amount causes an increase in sensitivity of the spores in the self-contained biological indicator to the oxidative sterilant in the sterilization process relative to an otherwise-identical self-contained biological indicator comprising a dry coating that lacks the effective amount of the sterilant-resistance modulator.

2. The self-contained biological sterilization indicator of claim 1, wherein the outer container comprises at least one wall, wherein at least a portion of the dry coating is disposed in the interior volume on the at least one wall.

3. The self-contained biological sterilization indicator of claim 1, further comprising a carrier, wherein at least a portion of the dry coating is disposed on the carrier.

4. The self-contained biological sterilization indicator of claim 1, wherein the dry coating is disposed in vapor communication with the atmosphere outside the container.

5. The self-contained biological sterilization indicator of claim 1, further comprising a means for forming a detectable indication of the failure of a sterilization procedure.

6. (canceled)

7. The self-contained biological sterilization indicator of claim 1, wherein the dry coating comprises about 10³ viable spores to about 10⁸ viable spores.

8. The self-contained biological sterilization indicator of claim 1, wherein the effective amount is about 2 micrograms to about 20 micrograms.

9. The self-contained biological sterilization indicator of claim 1, wherein the effective amount is about 0.02 nanograms/viable spore to about 0.2 nanograms/viable spore.

10. (canceled)

11. The self-contained biological sterilization indicator of claim 1, wherein the pathway is configured to hinder passage of a microorganism through the pathway.

12-16. (canceled)

17. A method of determining an efficacy of a sterilization process, the method comprising:
providing the self-contained biological sterilization indicator of claim 1;
exposing the self-contained biological sterilization indicator to a sterilant in a sterilization process, wherein the sterilant is a hydrogen peroxide sterilant; and
detecting an indication whether at least one of the plurality of spores survived the sterilization process.

18. (canceled)

19. The method of claim 17, wherein detecting an indication whether at least one of the plurality of spores survived the sterilization process comprises detecting growth of the spore.

20. The method of claim 17, wherein detecting an indication whether at least one of the plurality of spores survived the sterilization process comprises detecting a predetermined enzyme activity associated with the spore.

21. The self-contained biological sterilization indicator of claim 1,
wherein the increase in sensitivity of the spores is determined by exposing a first set of self-contained biological indicators to a first exposure to hydrogen peroxide sterilant and by exposing a second set of self-contained biological indicators to second exposure to hydrogen peroxide sterilant and subsequently activating the first and second sets of biological indicators and observing them for evidence of growth;
wherein the first and second sets each comprise a plurality of self-contained biological indicators comprising the sterilant-resistance modulator together with an equal plurality of otherwise-identical biological indicators not comprising the sterilant-resistance modulator;
wherein neither the first exposure nor the second exposure results in all of the biological indicators in the first set and second sets being growth-positive after activation;
wherein neither the first exposure nor the second exposure results in all of the biological indicators in the first and second sets being growth-negative after activation;
wherein the increase in sensitivity of the viable Geobacillus stearothermophilus spores is evidenced by a first percentage of growth-positive biological indicators comprising the sterilant-resistance modulator in the first set that is lower than a second percentage of growth-positive biological indicators not comprising the sterilant-resistance modulator in the first set and by a third percentage of growth-positive biological indicators comprising the sterilant-resistance modulator in the second set that is lower than a fourth percentage of growth-positive biological indicators not comprising the sterilant-resistance modulator in the second set.
